(19) United States
(12) Patent Application Publication
(10) Pub. No.: US 2015/0250514 A1
(43) Pub. Date: Sep. 10, 2015

(54) BONE FIXATION

(71) Applicant: COORSTEK MEDICAL LLC d/b/a IMDS, Providence, UT (US)

(72) Inventors: Lance Nathan Terrill, Oviedo, FL (US); Sarah Lynne Lancianese, Orlando, FL (US); Tyler Salem, Orlando, FL (US); Dain Odegaard, Orlando, FL (US)

(73) Assignee: COORSTEK MEDICAL LLC d/b/a IMDS, Providence, UT (US)

(21) Appl. No.: 14/203,462

(22) Filed: Mar. 10, 2014

(51) Int. Cl. A61B 17/86

(52) U.S. Cl. CPC A61B 17/8625

(57) ABSTRACT

Disclosed bone screws include a slotted tip. The slotted design of the tip partially obviates the need for precision while implanting the bone screw. Further, the slotted tip reduces the stiffness differential between the tip of the bone screw and the bone. Yet further, the slotted tip reduces stress concentrations imparted to the bone both during insertion and under bending loads. Moreover, the slotted tip conforms to natural formations within the bone when the bone screw is inserted into the bone. Instrumentation for implanting and orienting the bone screws is also disclosed herein.

### TECHNICAL FIELD

The present disclosure relates to systems, apparatuses, methods, and kits for bone fracture and/or joint repair. Specifically, this disclosure relates to a Jones fracture fixation apparatuses, systems, kits, and methods suitable to accommodate or correct various patient deformities.

### BACKGROUND

Jones fracture is a fracture of the diaphysis of the fifth metatarsal of the foot. The Jones fracture area is weak because of lack of blood flow to that area of the bone. In addition, the bone healing relies upon good circulation; therefore, it is challenging to treat the Jones fracture. The Jones fracture can be either a tiny hairline break that occurs over time (a stress fracture) or a sudden acute break. The Jones fracture is often caused by overuse, repetitive stress, or trauma.

There are two main options for the treatment of a Jones fracture: non-operative and surgical. The non-operative option involves wearing a walking boot for some weeks to let the bone heal on its own. However, as the Peroneus Brevis Muscle constantly puts the bone in tension, natural healing is difficult. Therefore, the surgical option is often used to aid compression at the fracture site to force a union. Surgically, the main options include a bone screw, a bone plate, a bone staple, and external fixators. A bone screw is the most common solution to repair the fracture. Bone screws have threads and a head opposed to the threads that allows the surgeon to apply the screw across a bony fracture. When the fracture is healed, the bone screw may be removed.

Specialized bone screws have been created for fixing Jones fractures. These screws are implanted in the center of the intramedullary canal of the fifth metatarsal. However, implantation is challenging because of the shape of the fifth metatarsal bone. The fifth metatarsal has a lateral bow on the dorsoplanter plane and a dorsal bow on the medolateral plane. In addition, the bone is irregular and shaped like a pyramid in the vertical cross-section. Therefore, screw sizing is very important. Targeting of the distal tip of the screw is also difficult. A misaligned screw could cause high stress concentrations on the inside of the intramedullary canal. In addition, even well-aligned screws have a much higher stiffness than the bone, resulting in elevated stress levels in the bone around the tip of the screw. Therefore, existing systems and procedures for Jones fracture repair may not be as effective as desired.

### SUMMARY

The present disclosure relates to bone and joint fixation, and instrumentation and methods for preparation and implantation of devices. Bone repair and/or joint fixation may be necessary in cases of fracture, pain and inflammation due to cartilage degeneration, nerve impingement, spinal misalignment, and motion instability. The primary examples described herein illustrate how this concept is applied to repairing a Jones fracture, but this concept applies equally to other fractures and joints. The disclosed bone screw is used for stabilizing a joint or a bone fracture. The bone screw includes a screw head at one end and a slotted tip at the other end. The slotted tip includes a plurality of slots. Further, the bone screw includes a shaft between the head and the tip, with the shaft having a threaded segment at the distal end. The slotted design of the tip partially obviates the need for precision in Jones fracture repair. The slotted tip helps reduce the stiffness differential between the tip of the bone screw and the bone. Further, it reduces stress concentrations imparted to the bone both during insertion and under bending loads. Moreover, the slotted tip conforms to the intramedullary canal of the fifth metatarsal as the bone screw is inserted into the bone to repair a Jones fracture.

According to an embodiment of the present disclosure, the slotted tip of a bone screw has three slots resulting in three arms, a shaft diameter equal to the major diameter of the bone screws, cortical style threads, a non-cannulated solid core, a low profile head, and a hexalobe drive connection. The three arms extend only partially along the length of the threads of the bone screw and have stress relief arcs at the base of each arm.

Those of skill in the art will recognize that the following description is merely illustrative of the principles of the disclosure, which may be applied in various ways to provide many different alternative embodiments and may be applicable outside the fields of surgery or medical devices. While the present disclosure is made in the context of Jones fracture for the purposes of illustrating the concepts of the design, it is contemplated that the present design and/or variations thereof may be suited to other uses, such as to support other joints in the human body and to stabilize bone fractures. Moreover, the implants, instrumentation, and methods set forth herein may be used in open, percutaneous, and/or minimally invasive procedures. All changes that come within the meaning and range of equivalency of the claims are to be embraced within their scope.

### BRIEF DESCRIPTION OF THE DRAWINGS

Various embodiments of the present disclosure will now be discussed with reference to the appended drawings. It will be appreciated that these drawings depict only typical examples of the present disclosure and are, therefore, not to be considered limiting of its scope.

FIG. 1 is a side view of a bone screw in accordance with one example of the present disclosure;

FIG. 2 is an isometric view of the bone screw in FIG. 1;

FIG. 3A is a cross-section view of the bone screw in FIG. 1 taken along the lines of 3A-3A of FIG. 1;

FIG. 3B is an enlarged view of a portion “3B” marked in FIG. 3A;

FIG. 4 shows a bottom view of the bone screw in FIG. 1;

FIG. 5 shows a top view of the bone screw in FIG. 1;

FIG. 6A shows side view of a driver to drive the bone screw in FIG. 1;

FIG. 6B is a cross-section view of the driver in FIG. 6A taken along the lines of 6B-6B of FIG. 6A;

FIG. 6C is a cross-section view of the driver in FIG. 6A taken along the lines of 6C-6C of FIG. 6A;

FIG. 7A is a side view of a tap for tapping a hole drilled for providing clearance to the bone screw of FIG. 1;

FIG. 7B is a cross-section view of the tap in FIG. 7A taken along the lines of 7B-7B of FIG. 7A;

FIG. 7C is a cross-section view of the tap in FIG. 7A taken along the lines of 7C-7C of FIG. 7A;

FIG. 7D is an enlarged view of a portion “7D” marked in FIG. 7B;

FIG. 8A shows a portion of a foot with the bone screw of FIG. 1 inserted in a bone according to an example of the present disclosure;

FIG. 8B shows an enlarged view of a portion marked in FIG. 8A; and

FIG. 9 is a flowchart illustrating a method for inserting the bone screw in FIG. 1, according to one aspect of the present disclosure.

### DETAILED DESCRIPTION

While certain embodiments are shown and described in detail below by way of illustration only, it will be clear to the person skilled in the art upon reading and understanding this disclosure that changes, modifications, and variations may be made and remain within the scope of the technology described herein. Further, while various features are grouped together in the embodiments for the purpose of streamlining the disclosure, it is appreciated that features from different embodiments may be combined to form additional embodiments that are all contemplated within the scope of the disclosed technology.

Not every feature of each embodiment is labeled in every figure where that embodiment appears, in order to keep the figures clear. Similar reference numbers (for example, those that are identical except for the first numeral) may be used to indicate similar features in different embodiments.

Any of the devices described herein may be fabricated from metals, alloys, polymers, plastics, ceramics, glasses, composite materials, or combinations thereof, including but not limited to: PEEK, titanium, titanium alloys, commercially pure titanium grade 2, ASTM F67, Nitinol, cobalt chrome, stainless steel, UHMWPE, and biodegradable materials, among others. Different materials may be used within a single part. The implants disclosed herein may also encompass a variety of surface treatments or additives to encourage bony attachment, including but not limited to: porous coatings, hydroxyapatite, TCP, anti-microbial additives, analgesics, anti-inflammatories, BMP's, PMA material, bone growth promoting material, PLLA (poly-L-lactide), PGA (polyglycolide), TCP (tricalcium phosphate), demineralized bone, cancellous bone chips, etc. Any implant disclosed herein may include a radiographic marker for imaging purposes. Any implant disclosed herein may be colored, coded, or otherwise marked to make it easier for the surgeon to identify the type and size of the implant.

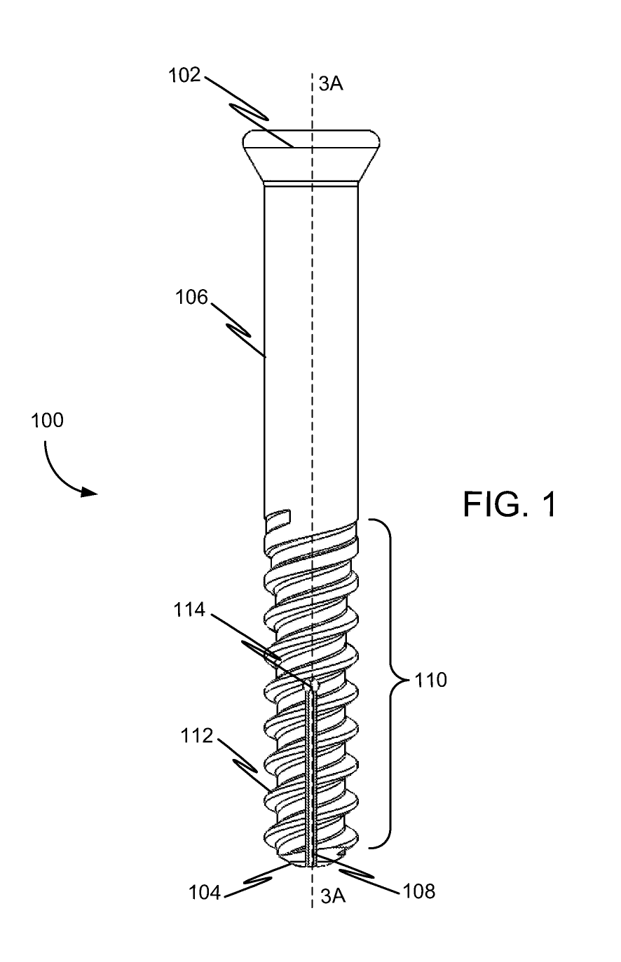

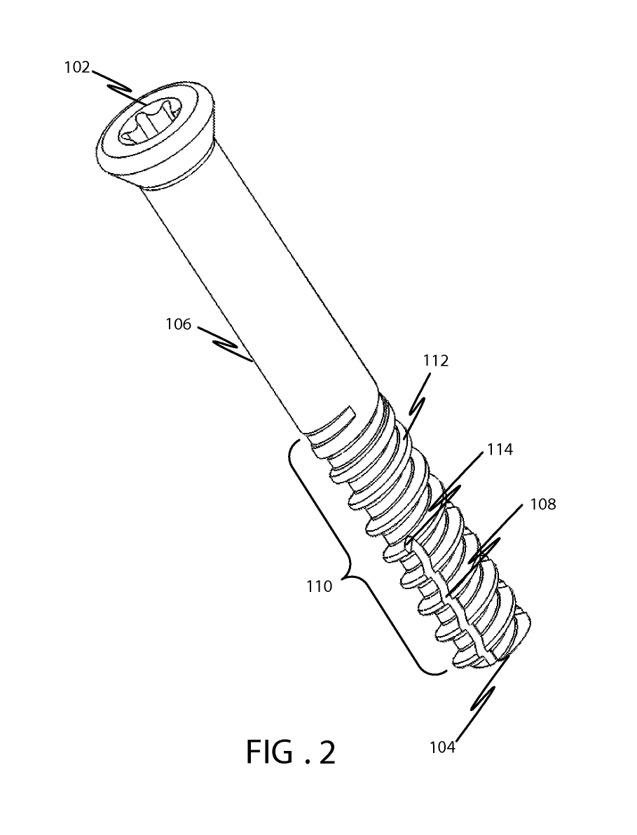

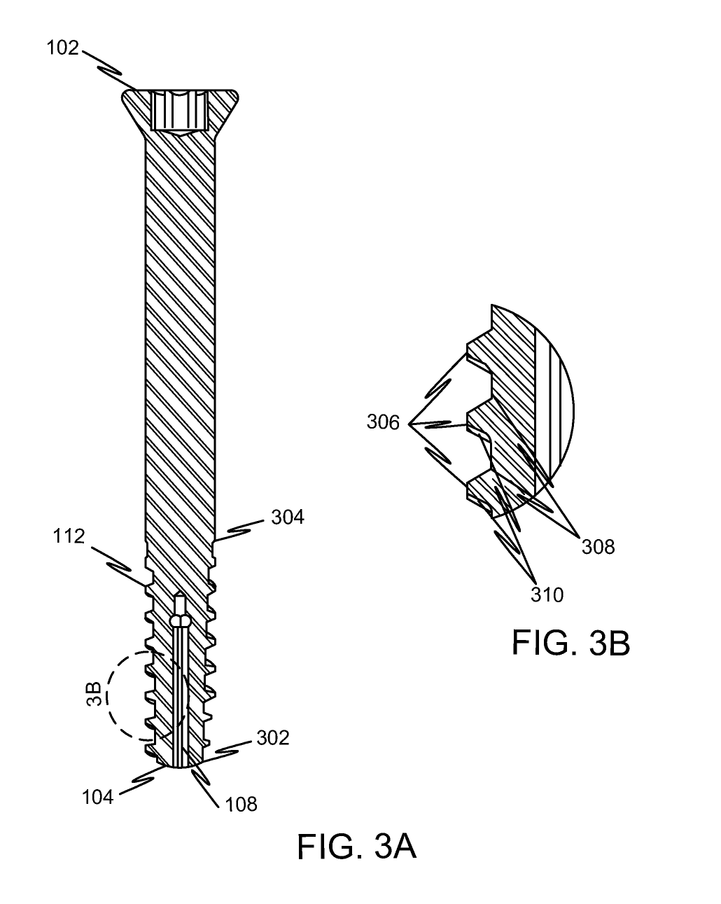

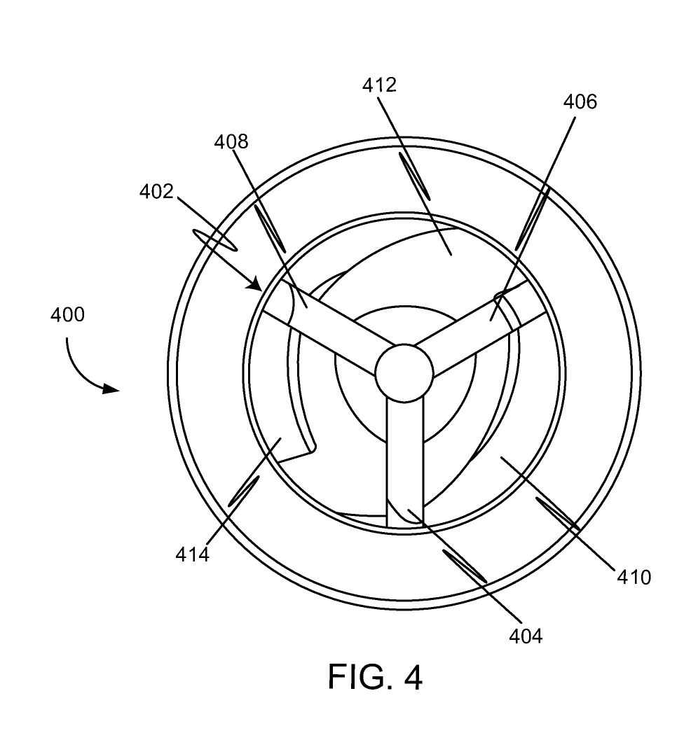

FIG. 1 illustrates one example of a bone screw 100 useful for stabilizing a joint or a bone fracture. FIG. 2 is an isometric view of the bone screw in FIG. 1. FIG. 3A is a cross-section view of the bone screw in FIG. 1 taken along the lines of 3A-3A of FIG. 1. The bone screw 100 includes a screw head 102 at one end, a slotted tip 104 at the other end, and a shaft 106 between the screw head 102 and the slotted tip 104. The screw head 102 has a low profile, which helps in reducing irritation to surrounding soft tissue. The slotted tip 104 includes a plurality of slots (a slot 108 is shown in FIG. 1). The slots extend only partially along the length of a threaded portion 110 of the bone screw 100, from a distal end of the slotted tip 104 to a proximal end 114 of the slots 108. In another embodiment, the slotted tip 104 has three slots as shown in FIG. 4. This is explained in more detail in conjunction with FIG. 4 below.

Further, the diameter of the shaft 106 is equal to the major diameter of the bone screw 100. The bone screw 100 may have fully threaded shaft or a thread configuration with a lag (as shown in the FIGS. 1-3). Accordingly, the shaft 106 includes the threaded portion 110 at the distal end. The threaded portion 110 may partially taper toward the slotted tip 104. Further, the threaded portion 110 includes an external thread 112, which allows the bone screw 100 to have a threaded engagement with the bone. The external thread 112 may have cortical style threads, which are closely-spaced, shallow threads that help better grip with the bone. When used to fix the Jones fracture, the external thread 112 engages with the fifth metatarsal bone of the foot.

FIG. 3B illustrates an enlarged portion marked “3B” in FIG. 3A. The external thread 112 is a helical thread which runs from a distal end 302 to a proximal end 304. The external helical thread 112 may be right or left handed. The external helical thread 112 further comprises an inner diameter and an outer diameter. The outer diameter is defined by the diameter of the circle obtained in a cross-sectional view taken at the ridges 306 in a plane containing the axis of the threads. The outer diameter engages with the bone during insertion. Similarly, the inner diameter is defined by the diameter of the circle obtained in a cross-sectional view taken at the valleys 308 in a plane containing the axis of the threads. The thread pitch may be increased or decreased depending on the mechanical needs for the application. Further, the thread pitch can be constant or variable. The ridges 306 and the valleys 308 include beveled surfaces 310 in between. The angle of the bevel is not critical to the present disclosure.

Referring now to FIG. 4 is a bottom view of a bone screw 400 showing a slotted tip 402 according to another example of the present disclosure. The slotted tip 402 has three slots 404, 406, and 408, resulting in formation of three arms 410, 412, and 414. The slotted design of the tip 402 partially obviates the need for precision in Jones fracture repair. Further, the slotted tip 402 reduces the stiffness differential between the tip of the bone screw and the bone. Further, the three arms 410, 412, and 414 have stress relief arcs at the base, which help reduce stress concentrations imparted to the bone both during insertion. Yet further, the base of each arm may be self-cutting, wherein the base of each arm has the form of one of a trocar and a radial cutting groove.

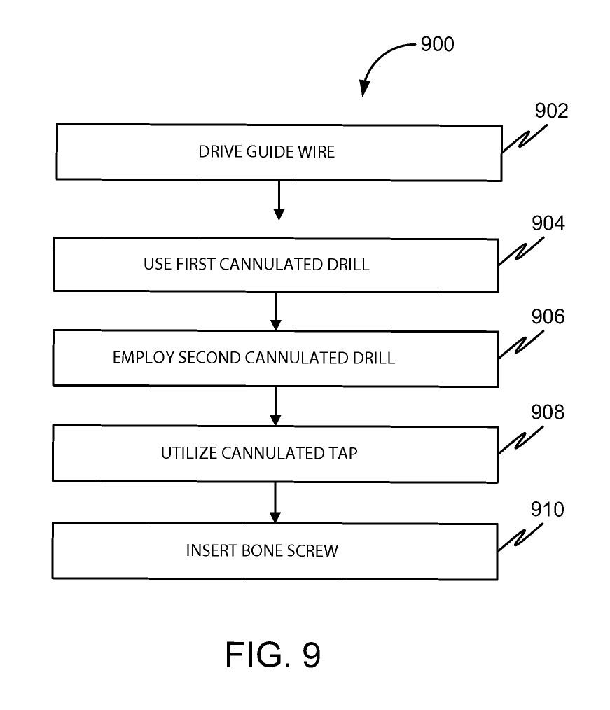

Referring back to FIG. 1, the bone screw 100 may have a cannulated core that is used to implant the bone screw 100 using a K-wire as explained in detail in conjunction with FIG. 9 below. Alternatively, the bone screw 100 has a non-cannulated solid core, as the bone screw 100 can be implanted without using a K-wire. In one treatment, a tiny incision is made on the skin on the outside of the foot and the bone screw 100 is inserted in a bone or in the intramedullary canal of the fifth metatarsalcanal (when repairing a Jones fracture). The bone screw 100 maintains compression between the bone segments and helps speed up the healing process.

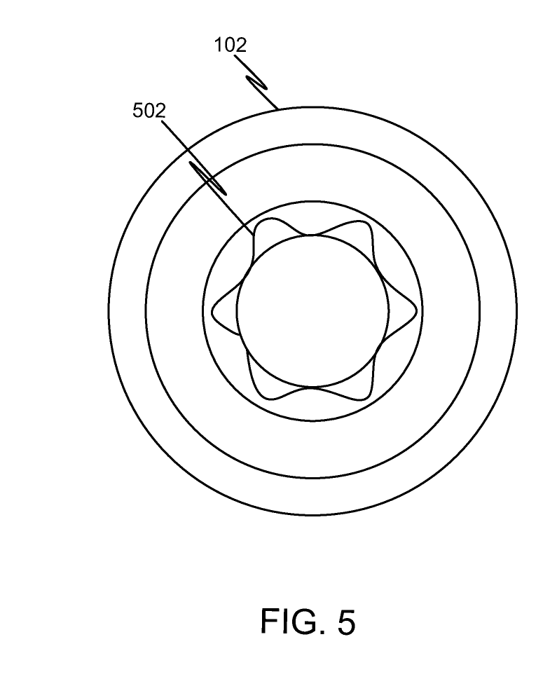

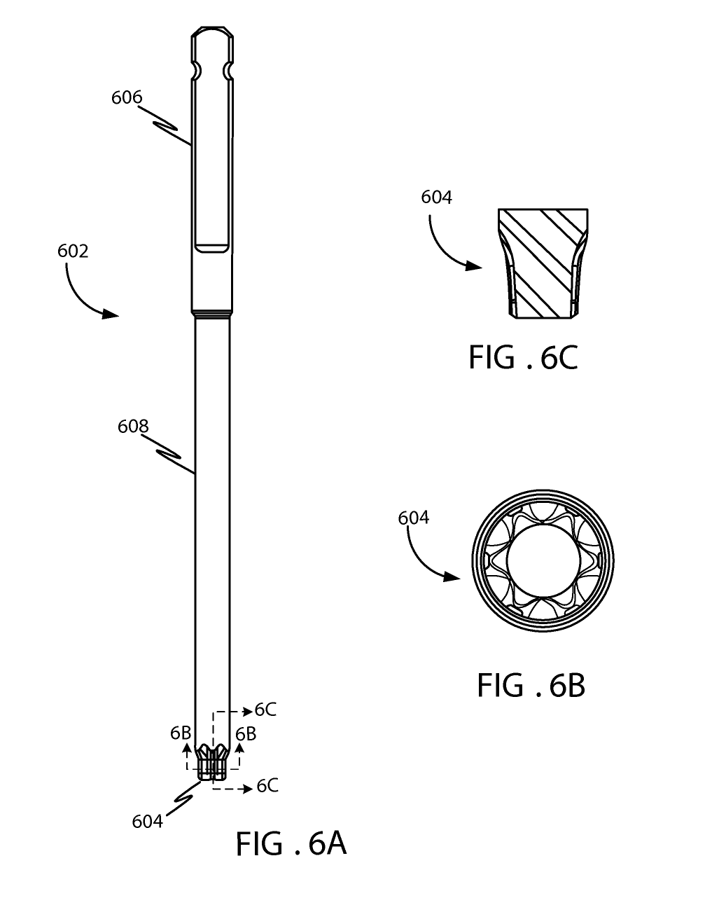

Further, the bone screw 100 may have a hexalobe drive connection 502 as shown in FIG. 5. Accordingly, a driver 602 with a hexalobe bit 604 as shown in FIGS. 6A and 6B may be used to implant the bone screw 100 in the bone. The hexalobe drive connection 502 provides a secure connection, preventing the driver 602 from stripping the bone screw 100. The hexalobe drive connection 502 also increases surface contact and reduces wear on the hexalobe bit 604. Therefore, the use of the driver 602 provides an accurate, safe and effective approach to inserting screws during surgeries. Alternatively, the bone screw 100 may have one of the following drive connections, a pentalobe drive connection, a torx drive connection, a cruciate drive connection, and a straight drive connection. Further, on the basis of the drive connection, an appropriate drive is chosen to implant the bone screw 100 in the bone.

FIG. 6A shows a side view of the driver 602 that may be used to implant the bone screw 100 in the bone. The driver 602 further includes a handle 606 and a shaft 608. The hexalobe bit 604 is designed to mate with the hexalobe drive connection 502 of the bone screw 100. FIG. 6B shows a cross-section view of the hexalobe bit 604 taken along the lines of 6B-6B of FIG. 6A. FIG. 6C shows a cross-section view of the hexalobe bit 604 taken along the lines of 6C-6C of FIG. 6A.

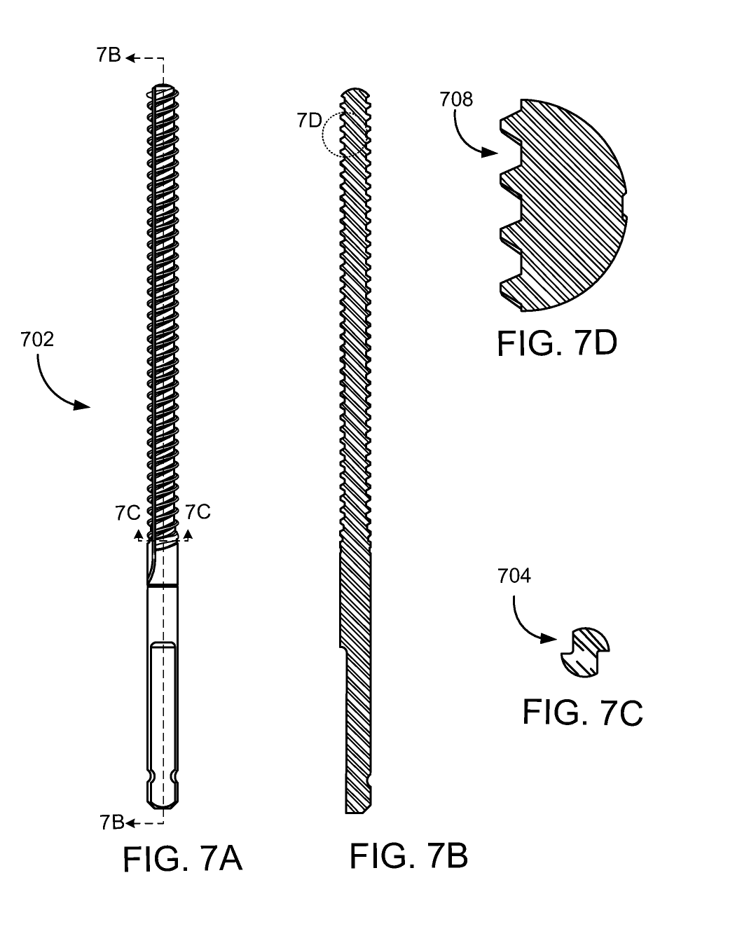

FIG. 7A shows a side view of a tap 702 that may be used to tap a hole drilled for providing clearance to the bone screw 100 in the bone. FIG. 7B is a cross-section view of the tap 702 in FIG. 7A taken along the lines of 7B-7B of FIG. 7A. FIG. 7C is a cross-section view of the tap in FIG. 7A taken along the lines of 7C-7C of FIG. 7A. FIG. 7 C shows cutting teeth 704 and 706, which are used to cut threads in the bone. FIG. 7D is an enlarged view of a portion marked “7D” in FIG. 7B showing a thread structure 708 of the tap 702. The thread structure 708 is designed to cut a thread in the bone that engages with the external thread 112 of the bone screw.

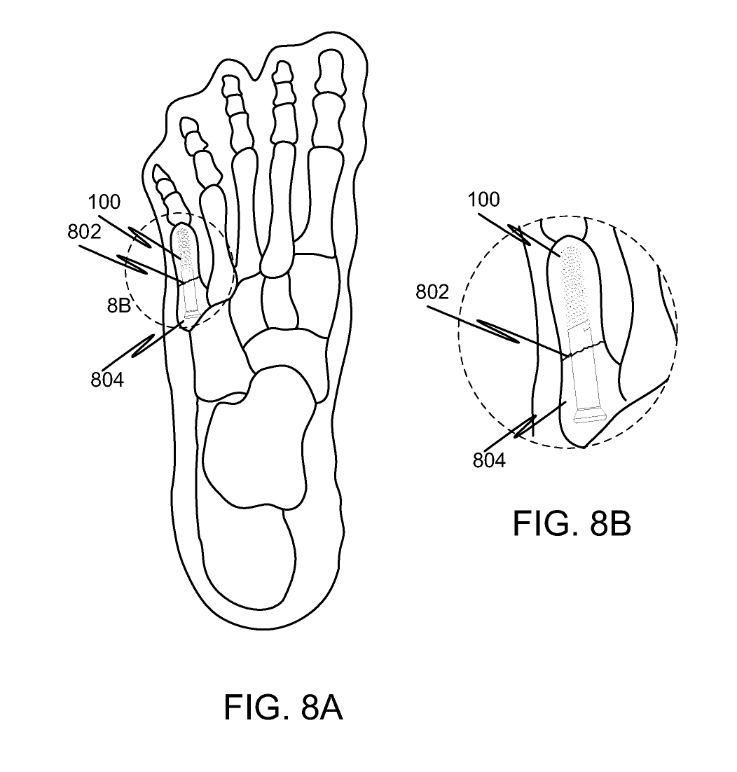

FIG. 8A shows an example of a placement of the bone screw 100 across a Jones fracture 802 in the fifth metatarsal 804. FIG. 8B shows an enlarged view of a portion marked “8B” in FIG. 8A. The shown placement of the bone screw 100 is easily achievable with the screw features, guides, drivers, and instrumentation disclosed herein. The external thread 112 engages with the fifth metatarsal 804.

Referring now to FIG. 9, depicting a method 900 of inserting the bone screw 100 into the bone as disclosed herein. As shown in the FIG. 8A, the bone screw 100 is implanted in the fifth metatarsal 804. At step 902, a surgeon inserts a guide wire (for example, a K-wire) to a desired location into the foot of a patient. When repairing Jones fracture, the K-wire is inserted such that it reaches the intramedullary canal of the fifth metatarsal. The surgeon may identify location of the bone by palpation and apply a target marking on the skin of the patient's body. The target marking identifies a desired position of the tip of the guide wire or a drill. Thereafter, the surgeon may use a targeting device that facilitates accurate placement of the bone screw 100 within the body of the patient. The targeting device may use one or more of a mechanical aid, a light source and an X-ray source to point toward the target location of placement of the bone screw 100. Once the guide wire is in the desired location, the surgeon may insert a dilator over the guide wire into the soft tissue of the patient to provide sufficient access to the bones. Once the tissue is dilated, the surgeon removes the dilator thus exposing the bones for the remainder of the surgery. Further, the surgeon may estimate the depth of the K-wire insertion.

At step 904, the surgeon guides a first cannulated drill over the guide wire to drill through the bone (e.g., the fifth metatarsal, when repairing a Jones fracture). The first cannulated drill provides clearance for threaded portion 110 (minor diameter). At step 906, the surgeon employs a second cannulated drill over the guide wire to drill through the bone and provide clearance for the shaft 106 (major diameter) of the bone screw 100. The first cannulated drill and the second cannulated drill may be manually operated or may be operated by, or as, powered devices, Thereafter, at step 908, the surgeon utilizes a cannulated tap to tap the hole drilled by the first cannulated drill and the second cannulated drill. Alternatively, the bone screw 100 may have a self-tapping (or self-threading) feature to tap a drilled hole when the bone screw 100 is inserted into the bone.

Once the implant site is sufficiently prepared to receive the bone screw 100, the surgeon uses the guide wire to orient and insert the bone screw 100 into the bone, at step 910. The surgeon uses the driver 602 to insert the bone screw 100 into the bone, and engages the threaded portion 110 with the bone. Further, the surgeon uses the driver 602 to achieve a required compression between the bones. Once the bone screw 100 is in the proper location, the surgeon can remove the guide wire, the drivers, and any other instrumentation used, and then close the incision site.

The present disclosure further provides a bone screw kit or system, which includes a plurality of bone screws, wherein each bone screw includes a screw head at one end, a slotted tip at the other end and a shaft between the head and tip, the shaft having a threaded segment at the distal end. The slotted tip includes plurality of slots. Moreover, each bone screw in the kit is of a different configuration, wherein the configuration of a bone screw is defined by one or more parameters including a number of slots, a major diameter, a pitch, a length, a lag thread, and a drive connection. In an embodiment, the kit includes screws in 0.5 mm major diameter increments; for example, diameters of bone screws may be 4.5 mm, 5.5 mm, and 6.5 mm. In the same or another embodiment, the length of the bone screws lies in the range of 20-70 mm. The bone screws may be made using titanium alloys in addition to any of the materials mentioned herein. The bone screw kit may further include one or more of a K-wire, one or more drilling tools, one or more tapping tools capable of accommodating all bone screws in the kit, and one or more driving tools.

It should be understood that the present components, systems, kits, apparatuses, and methods are not intended to be limited to the particular forms disclosed. Rather, they are intended to include all modifications, equivalents, and alternatives falling within the scope of the claims. They are further intended to include embodiments which may be formed by combining features from the disclosed embodiments, and variants thereof.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains,” one or more steps or elements, possesses those one or more steps or elements, but is not limited to possessing only those one or more elements. Likewise, a step of a method or an element of a device that “comprises,” “has,” “includes,” or “contains” one or more features, possesses those one or more features, but is not limited to possessing only those one or more features. Furthermore, a device or structure that is configured in a certain way is configured in at least that way, but may also be configured in ways that are not listed.

The present invention may be embodied in other specific forms without departing from its spirit or essential characteristics. It is appreciated that various features of the above-described examples can be mixed and matched to form a variety of other alternatives. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A bone screw for stabilizing a joint or a bone fracture, the bone screw comprising:
a screw head at one end;
a slotted tip at the other end, the slotted tip including a plurality of slots; and
a shaft between the head and tip, the shaft having a threaded segment at the distal end.

2. The bone screw of claim 1, wherein the bone screw is used for fixation of a Jones fracture.

3. The bone screw of claim 1, wherein the shaft diameter is equal to the major diameter of the screw.

4. The bone screw of claim 1, wherein the bone screw has a lag thread configuration.

5. The bone screw of claim 1, wherein the plurality of slots includes three slots, resulting in three arms at the slotted tip.

6. The bone screw of claim 5, wherein the three slots extend only partially along the length of the threaded segment.

7. The bone screw of claim 6, wherein base of each arm includes a stress relief arc.

8. The bone screw of claim 6, wherein the base of each arm is self-cutting.

9. The bone screw of claim 8, wherein the base of each arm has the form of one of a trocar and a radial cutting groove.

10. The bone screw of claim 1, wherein the threaded segment includes cortical style threads.

11. The bone screw of claim 1, wherein the threaded segment includes self-cutting threads.

12. The bone screw of claim 1, wherein the screw head has a low profile.

13. The bone screw of claim 1, wherein the screw head includes a drive connection feature including one of a pentalobe drive connection, a hexalobe drive connection, a torx drive connection, a cruciate drive connection, and a straight drive connection.

14. A kit comprising a plurality of bone screws according to claim 1, wherein each bone screw in the kit is of a different configuration, wherein the configuration of a bone screw is defined by one or more parameters including a number of slots at the tip, a major diameter, a pitch, a length, a lag thread, a drive connection, wherein the kit further comprises one or more of a K-wire, one or more drilling tools, one or more tapping tools capable of accommodating all bone screws in the kit, and one or more driving tools.

15. A method for stabilizing a joint or a bone fracture, the method comprising:
providing a bone screw comprising:
a screw head at one end;
a slotted tip at the other end, the slotted tip including a plurality of slots; and
a shaft between the screw head and the slotted tip, the shaft having a threaded segment at the distal end;
driving a guide wire to a desired position;
using a first cannulated drill to provide clearance for a minor diameter of the bone screw;
employing a second cannulated drill to provide clearance for the shaft of the bone screw;
utilizing a cannulated tap to tap threads for the bone screw; and
inserting the bone screw into the bone.

16. The method of claim 18, wherein the method is used for fixation of a Jones fracture.

17. The method of claim 18, wherein the bone screw includes three slots at the distal end, resulting in three arms at the tip.

18. An apparatus for stabilizing a joint or a bone fracture, the apparatus comprising:
a bone screw comprising:
a low profile screw head at one end;
a slotted tip at the other end, the slotted tip including a plurality of slots, wherein base of each slot includes a stress relief arc;
a shaft between the head and tip, the shaft having a threaded segment at the tip end, and wherein the threaded segment includes cortical style threads; and
a targeting device to facilitate accurate placement of the bone screw within a bone.

19. The apparatus of claim 1, wherein the bone screw is used for fixation of the Jones fracture, wherein the targeting device uses one or more of a mechanical aid, a light source, and an X-ray source to point toward the target location of placement of the bone screw.

20. The apparatus of claim 1, wherein the diameter of the shaft diameter is equal to the major diameter of the bone screw, and wherein the screw head includes
a drive connection feature comprising one of a pentalobe drive connection, a hexalobe drive connection, a torx drive connection, a cruciate drive connection, and a straight drive connection, and wherein the slotted tip includes three slots, resulting in three arms at the tip, wherein the three arms extend only partially along the length of the threaded segment.
